(19) United States
(12) Patent Application Publication
(10) Pub. No.: US 2026/0203470 A1
(43) Pub. Date: Jul. 16, 2026

(54) METHOD FOR SPECIMEN PREPARATION, AND METHOD FOR CHARACTERIZING INTERNAL THREE-DIMENSIONAL DEFORMATION OF SPECIMENS

(71) Applicant: CHINA UNIVERSITY OF MINING AND TECHNOLOGY, BEIJING, Beijing (CN)

(72) Inventors: Yang JU, Beijing (CN); Qing QIAO, Beijing (CN); Lingtao MAO, Beijing (CN); Zhangyu REN, Beijing (CN); Yun LEI, Beijing (CN)

(73) Assignee: CHINA UNIVERSITY OF MINING AND TECHNOLOGY, BEIJING, Beijing (CN)

(21) Appl. No.: 19/445,417

(22) Filed: Jan. 9, 2026

(30) Foreign Application Priority Data
Jan. 14, 2025 (CN) 202510051679.5

(51) Int. Cl. G06F 30/25; G01N 33/24; G06T 7/00; G06T 7/11; G06T 7/62; G06T 12/30

(52) U.S. Cl. CPC G06F 30/25; G01N 33/24; G06T 7/0002; G06T 7/11; G06T 7/62; G06T 12/30; G06T 2207/20056; G06T 2207/30144

(57) ABSTRACT

Methods for specimen preparation and for characterization of internal three-dimensional deformation of specimen are provided. Firstly, a plurality of first digital particle models are generated, and a plurality of first volumetric images are generated based on central point positions of the speckle particles simulated by each first digital particle model of the plurality of first digital particle models. A simulated optimal particle radius value and a simulated optimal volume fraction value corresponding to speckle particles included in a target specimen are determined based on all the first volumetric images. Finally, the target specimen may be prepared based on the simulated optimal particle radius value and the simulated optimal volume fraction value corresponding to the speckle particles included in the target specimen. The plurality of first digital particle models generated through numerical simulation are processed and calculated to obtain distribution parameters of the speckle particles in the target specimen.

### CROSS-REFERENCE OF RELATED APPLICATION

This application claims priority to Chinese Patent Application No. 202510051679.5, titled “Method for Specimen Preparation, and Method for Characterizing Internal Three-Dimensional Deformation of Specimens”, filed on Jan. 14, 2025 with the China National Intellectual Property Administration (CNIPA), which is incorporated herein by reference in its entirety.

### FIELD

The present disclosure relates to a technical field of mechanical measurement technologies, and particularly to a method for specimen preparation and a method for characterizing internal three-dimensional deformation of specimens.

### BACKGROUND

Precise preparation of rock specimens with complex structures and accurate measurement of three-dimensional deformation inside rock with complex structures are of great significance for researches on the mechanism of rock disasters. These researches can provide important reference for safe mining and disaster prevention and control.

Currently, in a laboratory, rock specimens with complex structures are mainly prepared by mechanical cutting and grinding a material such as natural rock, and three-dimensional deformation inside rock with complex structures is mainly characterized by Computed Tomography (CT) and Digital Volume Correlation (DVC). A rock specimen with a complex structure is prepared from natural rock material, and is subsequently subjected to CT scanning for analysis. Due to the presence of holes, cracks and various mineral components inside the natural rock, densities and X-ray absorption capacities at different positions of the rock specimen are different. During CT imaging, differences between the densities and the X-ray absorption capacities at different positions of the rock specimen are manifested as changes in intensity information on an image, and a natural speckle pattern would be formed inside the rock. By using DVC, changes in a position of the speckle pattern inside the specimen in the image may be tracked and calculated, to characterize the three-dimensional deformation inside the specimen.

However, due to heterogeneity and discontinuity of rock with complex structures, it is difficult to accurately and repeatedly prepare specimens with a consistent structure by using methods of mechanical cutting and grinding, and specimen preparation takes a long time. In addition, a speckle structure inside a specimen of a natural rock material is random. Some specimens even lack a speckle structure suitable for analysis, making it difficult to accurately obtain state changes inside the specimen during experiment. In addition, due to inevitable differences in internal structures of different specimens, it is difficult to summarize experimental results after repeating the experiment by using different specimens. Therefore, developing a method for specimen preparation with high stability, good repeatability and a short preparation cycle, and accurately characterizing three-dimensional deformation inside a prepared specimen is a key technical problem that needs to be solved urgently.

### SUMMARY

In view of this, one of technical problems solved by the present disclosure is to provide a method for specimen preparation and a method for characterizing internal three-dimensional deformation of specimens, to overcome a problem in the related art that a preparation cycle of specimens prepared from a natural rock material is long, complex structures inside different specimens are inconsistent, a speckle structure inside the specimen is random, and the accuracy of analyzing an internal state of the specimen using Digital Volume Correlation (DVC) is affected.

In a first aspect, embodiments of the present disclosure provides a method for specimen preparation, including:
generating a plurality of first digital particle models, where voxel regions simulated by all the first digital particle models are of a same size, and particle radius values of speckle particles simulated by all the first digital particle models and/or volume fraction values of speckle particles simulated by all the first digital particle models are different;
generating a plurality of first volumetric images based on central point positions of the speckle particles simulated by each first digital particle model of the plurality of first digital particle models, where the first volumetric image is a Gaussian speckle volumetric image;
determining a simulated optimal particle radius value and a simulated optimal volume fraction value corresponding to speckle particles included in a target specimen based on all the first volumetric images; and
preparing the target specimen based on the simulated optimal particle radius value and the simulated optimal volume fraction value corresponding to the speckle particles included in the target specimen.

In an embodiment, the determining a simulated optimal particle radius value and a simulated optimal volume fraction value corresponding to speckle particles included in a target specimen based on all the first volumetric images includes:
performing translation transformation on all the first volumetric images separately, and calculating a root mean squared error value corresponding to each first volumetric image of the plurality of first volumetric images;
performing surface fitting on the root mean squared error values corresponding to all the first volumetric images, the particle radius values of the speckle particles and the volume fraction values of the speckle particles to obtain a first fitting surface; and
determining the simulated optimal particle radius value and the simulated optimal volume fraction value corresponding to the speckle particles included in the target specimen based on a minimum value of the root mean squared error values in the first fitting surface.

In an embodiment, the performing translation transformation on all the first volumetric images separately, and calculating a root mean squared error value corresponding to each first volumetric image of the plurality of first volumetric images includes:
calculating the root mean squared error value corresponding to each first volumetric image of the plurality of first volumetric images through a formula of:

$$RMSE = \sqrt{{( \frac{{\sum }_{s = 1}^{N} {d}_{s}}{N} - {d}_{0} )}^{2} + {\sum }_{s = 1}^{N} {( \frac{{\sum }_{s = 1}^{N} {d}_{s}}{N} - {d}_{s} )}^{2} / ( N - 1 )} ,$$

where N is a number of speckle particles in the first digital particle model, dₛ is a displacement calculation value of an s-th speckle particle calculated by Digital Volume Correlation (DVC), and d₀ is a sub-voxel displacement value of the first volumetric image.

In an embodiment, the determining a simulated optimal particle radius value and a simulated optimal volume fraction value corresponding to speckle particles included in a target specimen based on all the first volumetric images includes:
calculating a mean intensity gradient value corresponding to each first volumetric image of the plurality of first volumetric images;
performing surface fitting on the mean intensity gradient values corresponding to all the first volumetric images, the particle radius values of the speckle particles and the volume fraction values of the speckle particles to obtain a second fitting surface; and
determining the simulated optimal particle radius value and the simulated optimal volume fraction value corresponding to the speckle particles included in the target specimen based on a maximum value of the mean intensity gradient values in the second fitting surface.

In an embodiment, the method further includes:
scanning, by using a target imaging equipment, a test specimen to obtain a second volumetric image, where a matrix of the test specimen is embedded with speckle particles with multiple particle radius values;
calculating a particle equivalent speckle size value and a noise equivalent speckle size value corresponding to the speckle particles with multiple particle radius values included in the test specimen based on the second volumetric image; and
obtaining a physical optimal particle radius value corresponding to the target imaging equipment based on the particle equivalent speckle size value and the noise equivalent speckle size value corresponding to the speckle particles with multiple particle radius values included in the test specimen, where the preparing the target specimen based on the simulated optimal particle radius value and the simulated optimal volume fraction value corresponding to the speckle particles included in the target specimen includes:
preparing the target specimen based on the simulated optimal particle radius value and the simulated optimal volume fraction value corresponding to the speckle particles included in the target specimen, and the physical optimal particle radius value corresponding to the target imaging equipment.

In an embodiment, the preparing the target specimen based on the simulated optimal particle radius value and the simulated optimal volume fraction value corresponding to the speckle particles included in the target specimen, and the physical optimal particle radius value corresponding to the target imaging equipment includes:
generating a second digital particle model based on a preset shape of the target specimen, and the simulated optimal particle radius value and the simulated optimal volume fraction value corresponding to the speckle particles included in the target specimen;
scaling the second digital particle model based on the physical optimal particle radius value corresponding to the target imaging equipment to obtain a three-dimensional (3D) digital volume model; and
preparing the target specimen with a same shape and size as the 3D digital volume model.

In an embodiment, the preparing the target specimen with a same shape and size as the 3D digital volume model includes:
generating a third volumetric image based on central point positions of all speckle particles simulated by the 3D digital volume model and the physical optimal particle radius value corresponding to the target imaging equipment, where in the third volumetric image, regions where the speckle particles are located and a region where the matrix is located are distinguished by binarization; and
preparing, based on the third volumetric image, the target specimen with the same shape and size as the 3D digital volume model by 3D printing.

In an embodiment, the calculating a particle equivalent speckle size value and a noise equivalent speckle size value corresponding to the speckle particles with multiple particle radius values included in the test specimen based on the second volumetric image includes:
performing segmentation on the second volumetric image to obtain volumetric image regions corresponding to the speckle particles with multiple particle radius values included in the test specimen;
performing autocorrelation analysis on the volumetric image region corresponding to the speckle particles of each particle radius value respectively after Fourier transformation, to obtain an autocorrelation curve corresponding to the speckle particles of each particle radius value; and
determining, in a case of an autocorrelation coefficient in the autocorrelation curve corresponding to the speckle particles of each particle radius value being equal to a preset coefficient value, a width of the autocorrelation curve as the particle equivalent speckle size value corresponding to the speckle particles of each particle radius value.

In an embodiment, the calculating a particle equivalent speckle size value and a noise equivalent speckle size value corresponding to the speckle particles with multiple particle radius values included in the test specimen based on the second volumetric image includes:
performing segmentation on the second volumetric image to obtain volumetric image regions corresponding to the speckle particles with multiple particle radius values included in the test specimen, where volumetric image regions corresponding to the speckle particles of all particle radius values are of a same size, and each volumetric image region of the volumetric image regions includes images of speckle particles of only one particle radius value;
calculating a matrix equivalent speckle size value corresponding to a matrix in volumetric image regions respectively corresponding to the speckle particles of each particle radius value; and
obtaining the noise equivalent speckle size value corresponding to the speckle particles of each particle radius value based on the matrix equivalent speckle size value corresponding to the matrix.

In a second aspect, embodiments of the present disclosure provides a method for characterizing internal three-dimensional deformation of specimens, including:
performing scanning and imaging, by using a target imaging equipment, on the target specimen prepared based on the above method to obtain a first scanning image;
performing a loading experiment on the target specimen to deform the target specimen;
performing scanning and imaging, by using the target imaging equipment, on the deformed target specimen to obtain a second scanning image; and
calculating a displacement field and/or a strain field inside the target specimen based on the first scanning image and the second scanning image.

### BRIEF DESCRIPTION OF THE DRAWINGS

To explain the technical solutions in the embodiments of the present disclosure more clearly, the following briefly introduces the drawings required in the embodiments. The drawings described below are only some embodiments of the present disclosure. For those of ordinary skill in the art, other drawings may be obtained based on these drawings without creative efforts.

FIG. 1 is a schematic flowchart of a method for specimen preparation disclosed in Embodiment 1 of the present disclosure;

FIG. 2 is an effect schematic diagram of generating a fourth volumetric image and a first volumetric image based on a first digital particle model;

FIG. 3 is an effect schematic diagram of a first fitting surface;

FIG. 4 is an effect schematic diagram of a second fitting surface;

FIG. 5 is a schematic flowchart of a method for specimen preparation disclosed in Embodiment 2 of the present disclosure;

FIG. 6 is an effect schematic diagram of scanning a test specimen to generate a second volumetric image; and

FIG. 7 is a schematic flowchart of a method for characterizing internal three-dimensional deformation of specimens disclosed in Embodiment 3 of the present disclosure.

### DETAILED DESCRIPTION

The following clearly and completely describes the technical solutions in the embodiments of the present disclosure in combination with the drawings in the embodiments of the present disclosure. The described embodiments are only some, not all, of the embodiments of the present disclosure. Based on the embodiments in the present disclosure, all other embodiments obtained by those of ordinary skill in the art without creative efforts belong to the protection scope of the present disclosure.

It should be noted that the terms “first”, “second”, “third” and “fourth” in the description and claims of the present disclosure are used to distinguish different objects, not to describe a specific order. The terms “include” and “have” in the embodiments of the present disclosure and any variations thereof are intended to cover non-exclusive inclusion. For example, a process, method, device, product or equipment that includes a series of steps or units is not necessarily limited to those steps or units clearly listed, but may include other steps or units not clearly listed or inherent to these processes, methods, products or equipment.

### Embodiment 1

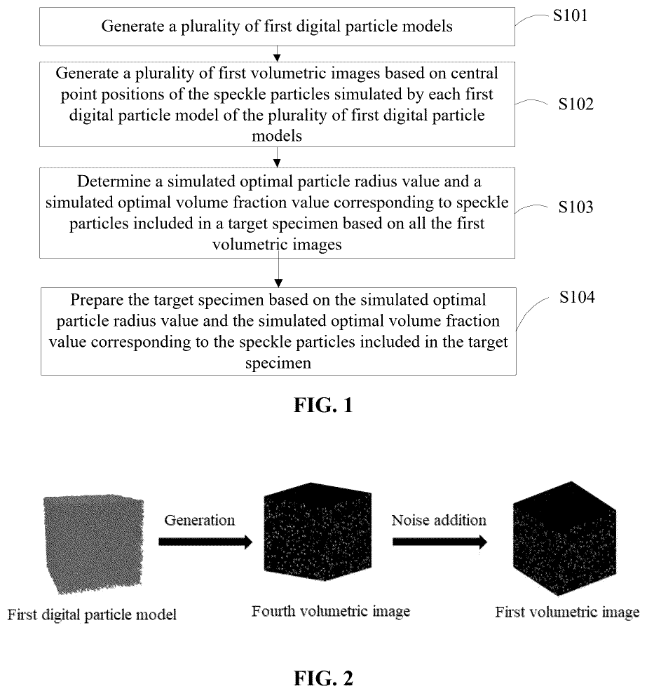

As shown in FIG. 1, FIG. 1 is a schematic flowchart of a method for specimen preparation disclosed in Embodiment 1 of the present disclosure. The method for specimen preparation includes the following steps.

In step S101, a plurality of first digital particle models are generated.

In this embodiment, the first digital particle model is a model generated by computer simulation software, used to simulate a specimen of a predetermined shape and size. Each simulated specimen includes a matrix and several speckle particles embedded in the matrix, where the speckle particles have the same or different shapes and sizes. That is, the particle radius values of all the speckle particles simulated by each first digital particle model of the plurality of first digital particle models may be the same or different, which is not limited in this embodiment.

The shapes and sizes of the specimens and speckle particles simulated by the first digital particle model are not limited, and may be reasonably selected based on actual application requirements. For example, on the premise of ensuring the accuracy of subsequent calculation results, it is preferable that all the first digital particle models are voxel regions of 100×100×100. To facilitate the preparation of speckle particles and the corresponding calculation and processing of volumetric image regions corresponding to the speckle particles, it is preferable that a shape of the speckle particles simulated by the first digital particle model is spherical.

In this embodiment, the voxel regions simulated by all the first digital particle models are of the same size, but the particle radius values and/or volume fraction values of the speckle particles simulated by different first digital particle models are different. That is, the particle radius values and/or volume fraction values of the speckle particles simulated by each first digital particle model of the plurality of first digital particle models are different compared with other first digital particle models.

In step S102, a plurality of first volumetric images are generated based on central point positions of the speckle particles simulated by each first digital particle model of the plurality of first digital particle models.

In this embodiment, the first volumetric image is a Gaussian speckle volumetric image. Based on the central point positions of all the speckle particles simulated by each first digital particle model of the plurality of first digital particle models, a corresponding first volumetric image may be generated. The first volumetric image may be a volumetric image added with Gaussian white noise or a volumetric image without added Gaussian white noise, which is not limited in this embodiment.

In an embodiment, referring to FIG. 2, to make the subsequent simulation calculation results closer to actual results, it is preferable that step S102 includes the following sub-step S102a and sub-step S102b.

In sub-step S102a, a plurality of fourth volumetric images are generated based on the central point positions of the speckle particles simulated by each first digital particle model of the plurality of first digital particle models.

The fourth volumetric image is an image directly generated based on the central point positions of the speckle particles simulated by each first digital particle model of the plurality of first digital particle models, and it is a volumetric image without adding Gaussian white noise.

In sub-step S102b, noise addition is performed on all fourth volumetric images based on a preset Gaussian white noise addition rule to obtain a plurality of first volumetric images.

The specific preset Gaussian white noise addition rule is not limited. The specific way of performing noise addition on all fourth volumetric images is not limited, and may be reasonably selected based on actual application requirements. For example, in order to perform noise addition more simply, it is preferable to add Gaussian white noise with a mean value of 0 and a standard deviation of 4% to each fourth volumetric image of the plurality of fourth volumetric images. In order to make the first volumetric image after noise addition more accurately represent a state of a physical specimen during Computed Tomography (CT) scanning, Gaussian noise estimation may be performed on the physical specimen to determine a mean value and standard deviation of the Gaussian white noise added to each fourth volumetric image of the plurality of fourth volumetric images.

In an embodiment, to reduce the complexity of calculation and improve the calculation accuracy of Digital Volume Correlation (DVC), it is preferable that the first volumetric image is a volumetric image without noise addition.

In step S103, a simulated optimal particle radius value and a simulated optimal volume fraction value corresponding to speckle particles included in a target specimen are determined based on all the first volumetric images.

In this embodiment, the target specimen is the specimen that finally needs to be prepared, that is, the specimen required for a loading experiment. Distribution parameters of the speckle particles in the target specimen may be determined based on all the first volumetric images, and then the target specimen may be prepared based on the distribution parameters of the speckle particles.

The distribution parameters of the speckle particles in the target specimen include the simulated optimal particle radius value and the simulated optimal volume fraction value corresponding to the speckle particles. The specific determination method of the simulated optimal particle radius value and the simulated optimal volume fraction value corresponding to the speckle particles included in the target specimen is not limited, and may be reasonably selected based on actual application requirements.

In an embodiment, to quickly and accurately determine the distribution parameters of the speckle particles in the target specimen, step S103 may include the following sub-step S103a to sub-step S103c.

In sub-step S103a, translation transformation is performed on all first volumetric images respectively, and a root mean squared error value corresponding to each first volumetric image of the plurality of first volumetric images is calculated.

Ways of performing translation transformation are the same for all the first volumetric images. Performing translation transformation on all first volumetric images respectively is equivalent to applying a same displacement value to specimens simulated by all first volumetric images. Displacement calculation values corresponding to several positions of each first volumetric image before and after translation transformation may be calculated through DVC, especially displacement calculation values of speckle particles before and after translation transformation.

In addition, the specific calculation method of the root mean squared error value corresponding to each first volumetric image of the plurality of first volumetric images is not limited, and may be reasonably selected based on actual application requirements.

In sub-step S103b, surface fitting is performed on the root mean squared error values corresponding to all the first volumetric images, the particle radius values of the speckle particles and the volume fraction values of the speckle particles to obtain a first fitting surface.

The first fitting surface is used to characterize a corresponding relationship among the root mean squared error value, the particle radius value of the speckle particles, and the volume fraction value of the speckle particles. A type of a specific function used to obtain the first fitting surface through fitting is not limited, and may be reasonably selected based on actual application requirements.

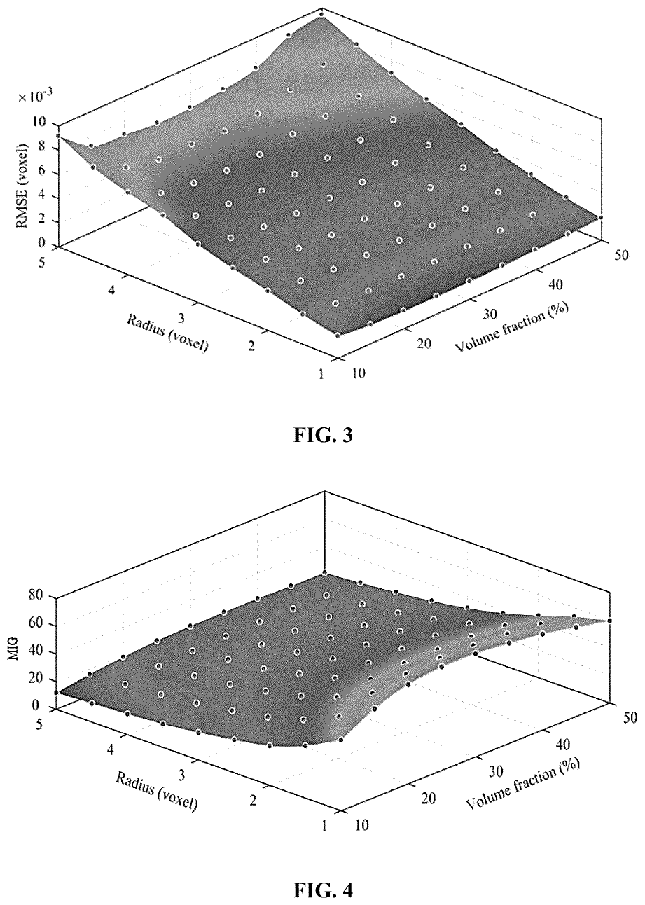

An effect schematic diagram of a first fitting surface may be referred to FIG. 3. In FIG. 3, RMSE corresponds to a root mean squared error value, radius corresponds to a particle radius value of a speckle particle, and volume fraction corresponds to a volume fraction value of a speckle particle.

In sub-step S103c, the simulated optimal particle radius value and the simulated optimal volume fraction value corresponding to the speckle particles included in the target specimen are determined based on a minimum value of the root mean squared error values in the first fitting surface.

After determining a position where the minimum value of the root mean squared error values is located in the first fitting surface, a particle radius value and a volume fraction value of speckle particles corresponding to the minimum value may be further determined and taken as the simulated optimal particle radius value and the simulated optimal volume fraction value corresponding to the speckle particles included in the target specimen.

Further, to calculate the root mean squared error value corresponding to each first volumetric image of the plurality of first volumetric images more accurately, sub-step S103a may also include:
obtaining the root mean squared error value corresponding to each first volumetric image of the plurality of first volumetric images by using the following formula:

$$RMSE = \sqrt{{( \frac{{\sum }_{s = 1}^{N} {d}_{s}}{N} - {d}_{0} )}^{2} + {\sum }_{s = 1}^{N} {( \frac{{\sum }_{s = 1}^{N} {d}_{s}}{N} - {d}_{s} )}^{2} / ( N - 1 )} ,$$

where N is a number of speckle particles in the first digital particle model, dₛ is a displacement calculation value of an s-th speckle particle calculated by Digital Volume Correlation (DVC), and d₀ is a sub-voxel displacement value of the first volumetric image.

In addition, in the above formula, all the speckle particles simulated by the first digital particle model may be selected to calculate the root mean squared error value corresponding to each first volumetric image of the plurality of first volumetric images, or some of the speckle particles simulated by the first digital particle model may be selected to calculate the root mean squared error value corresponding to each first volumetric image of the plurality of first volumetric images, which is not limited in this embodiment.

In an embodiment, to quickly and accurately determine the distribution parameters of the speckle particles in the target specimen, step S103 may further include the following sub-step S103d to sub-step S103f.

In sub-step S103d, a mean intensity gradient value corresponding to each first volumetric image of the plurality of first volumetric images is calculated.

In sub-step S103e, surface fitting is performed on the mean intensity gradient values corresponding to all the first volumetric images, the particle radius values of the speckle particles and the volume fraction values of the speckle particles to obtain a second fitting surface.

The second fitting surface is used to characterize a corresponding relationship among the mean intensity gradient value, the particle radius value of the speckle particle and the volume fraction value of the speckle particle. A type of a specific function used to obtain the second fitting surface through fitting is not limited, and may be reasonably selected based on actual application requirements.

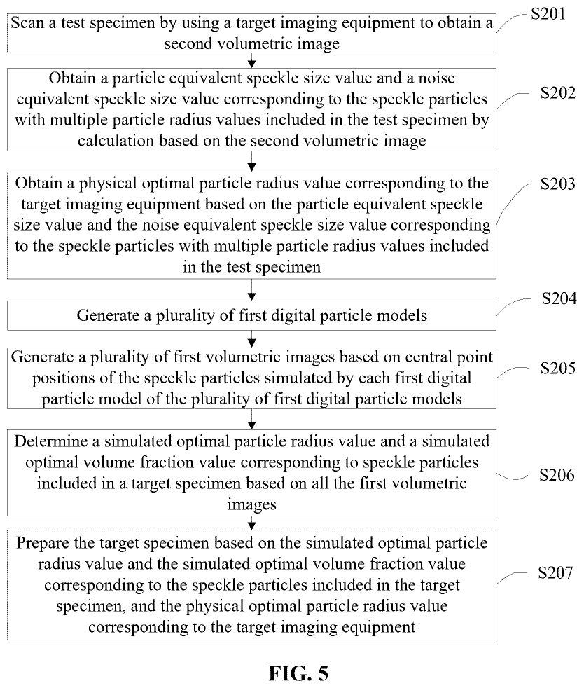

An effect schematic diagram of a second fitting surface may be referred to FIG. 4. In FIG. 4, MIG corresponds to the mean intensity gradient value, radius corresponds to the particle radius value of a speckle particle, and volume fraction corresponds to the volume fraction value of a speckle particle.

In sub-step S103f, the simulated optimal particle radius value and the simulated optimal volume fraction value corresponding to the speckle particles included in the target specimen are determined based on a maximum value of the mean intensity gradient values in the second fitting surface.

After determining a position where the maximum value of the mean intensity gradient values is located in the second fitting surface, a particle radius value and a volume fraction value of speckle particles corresponding to the maximum value may be further determined and taken as the simulated optimal particle radius value and the simulated optimal volume fraction value corresponding to the speckle particles included in the target specimen.

In addition, in this embodiment, sub-step S103a to sub-step S103c and sub-step S103d to sub-step S103f may be implemented alternatively, or both of them may be implemented to mutually verify the accuracy of the calculation of the simulated optimal particle radius value and the simulated optimal volume fraction value corresponding to the speckle particles.

In step S104, the target specimen is prepared based on the simulated optimal particle radius value and the simulated optimal volume fraction value corresponding to the speckle particles included in the target specimen.

In this embodiment, a specific material for preparing the target specimen and the method for preparing the target specimen are not limited, and may be reasonably selected based on actual application requirements, but it is necessary to ensure that the materials of the speckle particles and the matrix included in the target specimen are different.

In addition, in a case that the target specimen is imaged by rays during the preparation of the specimen, it is also necessary to ensure that the materials of the speckle particles and the matrix included in the target specimen have different absorption rates for the rays. For example, to make a difference in the absorption rates of the two materials of the speckle particles and the matrix included in the target specimen for rays larger, it is preferable that a preparation material of the matrix is VeroClear material, and a preparation material of the speckle particles is RadioMatrixmaterial.

As may be seen from the above embodiments of the present disclosure, in this embodiment, firstly, a plurality of first digital particle models are generated, and then a plurality of first volumetric images are generated based on central point positions of the speckle particles simulated by each first digital particle model of the plurality of first digital particle models. A simulated optimal particle radius value and a simulated optimal volume fraction value corresponding to speckle particles included in a target specimen are then determined based on all the first volumetric images. Finally, the target specimen may be prepared based on the simulated optimal particle radius value and the simulated optimal volume fraction value corresponding to the speckle particles included in the target specimen. In the embodiments of the present disclosure, the plurality of first digital particle models generated through numerical simulation are processed and calculated to obtain distribution parameters of the speckle particles in the target specimen. Compared with the related art, a speckle structure inside the prepared target specimen is more stable, which is beneficial to improving the accuracy of analyzing an internal state of the specimen using DVC.

### Embodiment 2

As shown in FIG. 5, FIG. 5 is a schematic flowchart of a method for specimen preparation disclosed in embodiment 2 of the present application. The method for specimen preparation includes the following steps.

In step S201, a test specimen is scanned by using a target imaging equipment to obtain a second volumetric image.

In this embodiment, the target imaging equipment is used to image a matrix and a speckle structure of the target specimen throughout the entire experiment to obtain a corresponding volumetric image. A specific type and performance parameters of the target imaging equipment are not limited, and may be reasonably selected based on actual application requirements.

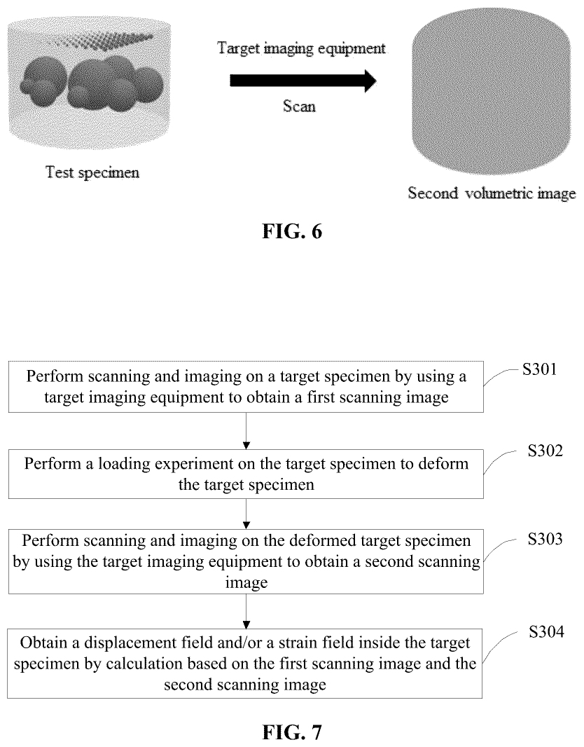

In this embodiment, the test specimen is a pre-prepared physical specimen. A matrix of the test specimen is embedded with speckle particles with multiple particle radius values. The number and particle radius values of the speckle particles in the test specimen are not limited, and may be reasonably selected based on actual application requirements. An effect schematic diagram about a test specimen and a second volumetric image may be referred to FIG. 6.

For example, to ensure the accuracy of subsequent calculation results and reduce data calculation amount, it is preferable that the matrix of the test specimen is embedded with speckle particles having 100 kinds of particle radius values, and particle radius values of all the speckle particles are distributed in an arithmetic sequence.

For another example, to reduce the data calculation amount, it is preferable that the number of speckle particles of each particle radius value is 1.

For a further example, to facilitate the preparation of the test specimen and ensure the imaging effect, it is preferable that a particle radius of the speckle particles in the test specimen ranges from 0.01 mm to 1 mm.

In step S202, a particle equivalent speckle size value and a noise equivalent speckle size value corresponding to the speckle particles with multiple particle radius values included in the test specimen are calculated based on the second volumetric image.

In this embodiment, the particle equivalent speckle size value is used to characterize an equivalent size of the speckle particles in the second volumetric image, and the noise equivalent speckle size value is used to characterize an equivalent size of the matrix in the second volumetric image. In experiments and researches, a volumetric image region where the speckle particles are located is a main research object, and a volumetric image region of a matrix without speckle particles would interfere with the research object. Therefore, to obtain a better imaging effect, the volumetric image region where the matrix is located may be regarded as a noise volumetric image region, and the noise equivalent speckle size value is calculated in step S202.

In this embodiment, to determine a particle radius value of speckle particles with an optimal imaging effect by the target imaging equipment from the speckle particles with multiple particle radius values, the particle equivalent speckle size value and the noise equivalent speckle size value corresponding to the speckle particles of each particle radius value may be calculated respectively.

A specific calculation method of the particle equivalent speckle size value and the noise equivalent speckle size value corresponding to the speckle particles of each particle radius value are not limited, and may be reasonably selected based on actual application requirements. For example, after segmenting the second volumetric image, the particle equivalent speckle size value and the noise equivalent speckle size value corresponding to the speckle particles of each particle radius value may be calculated based on the segmented volumetric image region. Alternatively, the particle equivalent speckle size value and the noise equivalent speckle size value corresponding to the speckle particles of each particle radius value may be calculated integrally by using a specific algorithm without segmenting the second volumetric image.

In an embodiment, to determine the particle equivalent speckle size value corresponding to the speckle particles of each particle radius value more accurately and reasonably, step S202 may further include the following sub-step S202a to sub-step S202c.

In sub-step S202a, segmentation is performed on the second volumetric image to obtain volumetric image regions corresponding to the speckle particles with multiple particle radius values included in the test specimen.

To compare imaging effects of the target imaging equipment for the speckle particles of different particle radius values more objectively, the volumetric image regions corresponding to the speckle particles of each particle radius value are the same, that is, the shapes and sizes of all segmented volumetric image regions are the same.

In addition, to reduce the data calculation amount, it is preferable that each volumetric image region includes only one speckle particle.

In sub-step S202b, autocorrelation analysis is performed on the volumetric image region corresponding to the speckle particles of each particle radius value respectively after Fourier transformation, to obtain an autocorrelation curve corresponding to the speckle particles of each particle radius value.

The Fourier transformation is used to convert the volumetric image from a spatial domain to a frequency domain.

In sub-step S202c, in a case of an autocorrelation coefficient in the autocorrelation curve corresponding to the speckle particles of each particle radius value being equal to a preset coefficient value, a width of the autocorrelation curve is determined as the particle equivalent speckle size value corresponding to the speckle particles of each particle radius value.

A specific value of the preset coefficient value is not limited, and may be reasonably selected based on actual application requirements. For example, to determine the particle equivalent speckle size value corresponding to the speckle particles of each particle radius value more accurately and objectively, it is preferable that the preset coefficient value is 0.5.

In an embodiment, to determine the noise equivalent speckle size value corresponding to the speckle particles of each particle radius value more accurately and reasonably, step S202 may further include the following sub-step S202d to sub-step S202f.

In sub-step S202d, segmentation is performed on the second volumetric image to obtain volumetric image regions corresponding to the speckle particles with multiple particle radius values included in the test specimen.

Volumetric image regions corresponding to the speckle particles of all particle radius values are of a same size, and each volumetric image region of the volumetric image regions includes images of speckle particles of only one particle radius value. In addition, to reduce the data calculation amount, it is preferable that each volumetric image region includes only one speckle particle.

In sub-step S202e, a matrix equivalent speckle size value corresponding to a matrix in volumetric image regions respectively corresponding to the speckle particles of each particle radius value is calculated.

A calculation method of the matrix equivalent speckle size value corresponding to the matrix is not limited, and may be reasonably selected based on actual application requirements. For example, volumetric image regions at several positions of the matrix in the volumetric image region may be calculated respectively, and the matrix equivalent speckle size value corresponding to the matrix may be determined based on calculation results.

Further, to obtain a more accurate calculation results, it is preferable that the matrix equivalent speckle size value corresponding to the matrix is calculated on volumetric image regions at a plurality of positions of the matrix respectively, and an average value of calculation results is determined as the matrix equivalent speckle size value corresponding to the matrix.

In sub-step S202f, the noise equivalent speckle size value corresponding to the speckle particles of each particle radius value is obtained based on the matrix equivalent speckle size value corresponding to the matrix.

The noise equivalent speckle size value corresponding to the speckle particles of each particle radius value in sub-step S202f may be the same as or different from the matrix equivalent speckle size value corresponding to the matrix in sub-step S202e, which is not limited in this embodiment. For example, the matrix equivalent speckle size value corresponding to the matrix may be directly used as the noise equivalent speckle size value corresponding to the speckle particles. Alternatively, the noise equivalent speckle size value corresponding to the speckle particles may be obtained by multiplying the matrix equivalent speckle size value corresponding to the matrix by a preset safety coefficient value.

Further, to more accurately measure interference of the volumetric image region of the matrix on the image region of the studied speckle particles, sub-step S202f may include: taking a product of the matrix equivalent speckle size value corresponding to the matrix and the preset safety coefficient value as the noise equivalent speckle size value corresponding to the speckle particles of each particle radius value.

The preset safety coefficient value usually needs to be greater than 1, and the specific value is not limited, and may be reasonably selected based on actual application requirements. For example, to obtain a more accurate and reasonable calculation result, it is preferable that the preset safety coefficient value is greater than or equal to 1.1 and less than or equal to 1.5.

In step S203, a physical optimal particle radius value corresponding to the target imaging equipment is obtained based on the particle equivalent speckle size value and the noise equivalent speckle size value corresponding to the speckle particles with multiple particle radius values included in the test specimen.

In this embodiment, imaging effects of the volumetric image region corresponding to the speckle particles of different particle radius values may be jointly measured based on the particle equivalent speckle size value and the noise equivalent speckle size value. A specific determination method of the physical optimal particle radius value corresponding to the target imaging equipment is not limited, and may be reasonably selected based on actual application requirements.

For example, a ratio of the particle equivalent speckle size value to the noise equivalent speckle size value may be referred to, and a particle radius value of the speckle particle with a smallest ratio among ratios greater than or equal to 1 may be determined as the physical optimal particle radius value corresponding to the target imaging equipment. Alternatively, fitting may be performed on the particle equivalent speckle size values and the noise equivalent speckle size values corresponding to the speckle particles of all particle radius values, and the physical optimal particle radius value corresponding to the target imaging equipment may be determined based on an obtained fitting curve.

In an embodiment, to simply and reasonably determine the physical optimal particle radius value corresponding to the target imaging equipment, step S203 may include the following sub-step S203a and sub-step S203b.

In sub-step S203a, among ratios of the particle equivalent speckle size value to the noise equivalent speckle size value, a speckle particle with a smallest ratio greater than or equal to 1 is determined as an optimal speckle particle.

In sub-step S203b, the physical optimal particle radius value corresponding to the target imaging equipment is obtained based on a particle radius value of the optimal speckle particle.

The particle radius value of the optimal speckle particle may be directly used as the physical optimal particle radius value corresponding to the target imaging equipment. The physical optimal particle radius value corresponding to the target imaging equipment may also be a value obtained by multiplying the particle radius value of the optimal speckle particle by the preset optimization coefficient value, which is not limited in this embodiment.

In step S204, a plurality of first digital particle models are generated.

In this embodiment, step S204 is basically the same or similar to step S101 in Embodiment 1, and would not be described in detail here.

In step S205, a plurality of first volumetric images are generated based on central point positions of the speckle particles simulated by each first digital particle model of the plurality of first digital particle models.

In this embodiment, step S205 is basically the same or similar to step S102 in Embodiment 1, and would not be described in detail here.

In step S206, a simulated optimal particle radius value and a simulated optimal volume fraction value corresponding to speckle particles included in a target specimen are determined based on all the first volumetric images.

In this embodiment, step S206 is basically the same or similar to step S103 in Embodiment 1, and would not be described in detail here.

In step S207, the target specimen is prepared based on the simulated optimal particle radius value and the simulated optimal volume fraction value corresponding to the speckle particles included in the target specimen, and the physical optimal particle radius value corresponding to the target imaging equipment.

In this embodiment, not only different types of imaging equipment may have certain differences in imaging effects of the target specimen, but also a same type of imaging equipment may have different imaging effects on the same target specimen due to differences in equipment performance parameters. Therefore, in order to better record a state change of the target specimen during the experiment, after determining the simulated optimal particle radius value and the simulated optimal volume fraction value corresponding to the speckle particles included in the target specimen, distribution parameters of the speckle particles of the target specimen may be determined in combination with the physical optimal particle radius value corresponding to the target imaging equipment, and the target specimen may be prepared further based on the distribution parameters of the speckle particles.

In an embodiment, to obtain a better specimen preparation effect, a three-dimensional (3D) digital volume model of the target specimen may be generated first, and then the target specimen may be prepared based on the 3D digital volume model. In an embodiment, step S207 may further include the following sub-step S207a to sub-step S207d.

In sub-step S207a, a second digital particle model is generated based on a preset shape of the target specimen, and the simulated optimal particle radius value and the simulated optimal volume fraction value corresponding to the speckle particles included in the target specimen.

The preset shape of the target specimen may be set based on actual application requirements, which is not specifically limited in this embodiment. For example, to facilitate processing and obtain a better imaging effect, the preset shape of the target specimen may be preferably a cylindrical shape.

In addition, the method for generating the second digital particle model in sub-step S207a is basically the same as the method for generating the first digital particle model in the step S204, the main difference is that an overall shape and/or an overall size of the two digital particle models may be different. However, the particle radius values and the volume fraction values corresponding to the speckle particles in the two models are the same when simulating speckle particles with the same size value and the same volume fraction value.

In sub-step S207b, the second digital particle model is scaled based on the physical optimal particle radius value corresponding to the target imaging equipment to obtain a 3D digital volume model.

The scaling of the second digital particle model includes making a size of the second digital particle model reduced, enlarged or keeping unchanged. The specific processing is mainly determined based on the physical optimal particle radius value corresponding to the target imaging equipment, which is not limited in this embodiment.

In sub-step S207c, the target specimen with a same shape and size as the 3D digital volume model is prepared.

Further, sub-step S207b may include: scaling the second digital particle model based on the physical optimal particle radius value corresponding to the target imaging equipment and a preset magnification value, to obtain the 3D digital volume model.

To ensure the imaging effect of the target imaging equipment on the target specimen, the preset magnification value may be preferably less than or equal to a minimum magnification value of the target imaging equipment.

Further, to prepare a target specimen including speckle particles with good stability and repeatability, the target specimen may be prepared by 3D printing. In an embodiment, sub-step S207c may further include the following sub-step A and sub-step B.

In sub-step A, a third volumetric image is generated based on central point positions of all speckle particles simulated by the 3D digital volume model and the physical optimal particle radius value corresponding to the target imaging equipment.

In the third volumetric image, a region where the speckle particles are located and a region where the matrix is located are distinguished by binarization. In addition, a complex three-dimensional structure, such as a complex fracture structure, may also be added to the third volumetric image through image processing technology.

In sub-step B, the target specimen with the same shape and size as the 3D digital volume model is prepared by 3D printing based on the third volumetric image.

As may be seen from the above embodiments of the present disclosure, compared with Embodiment 1, in this embodiment, a target specimen is prepared based on the simulated optimal particle radius value and the simulated optimal volume fraction value corresponding to the speckle particles, and the physical optimal particle radius value corresponding to the target imaging equipment, which can significantly improve an imaging effect of the target specimen by using the target imaging equipment. The target specimen being prepared by 3D printing not only has a short specimen preparation cycle, but also can obtain a target specimen including speckle particles with good stability and repeatability, which is beneficial to improving an experimental effect and accuracy.

### Embodiment 3

Embodiment 3 of the present application provides a method for characterizing internal three-dimensional deformation of specimens. FIG. 7 is a schematic flowchart of a method for characterizing internal three-dimensional deformation of a specimen disclosed in Embodiment 3 of the present application. The method for characterizing internal three-dimensional deformation of specimens includes the following steps.

In step S301, scanning and imaging are performed on a target specimen by using a target imaging equipment to obtain a first scanning image.

In this embodiment, the target specimen may be a specimen prepared by any optional implementation manner in Embodiment 1 and Embodiment 2.

In step S302, a loading experiment is performed on the target specimen to deform the target specimen.

In this embodiment, a specific way of performing the loading experiment on the target specimen is not limited and is mainly determined based on experimental requirements. For example, it may be an in-situ loading, ex-situ loading, etc.

In step S303, scanning and imaging are performed on the deformed target specimen by using the target imaging equipment to obtain a second scanning image.

In step S304, a displacement field and/or a strain field inside the target specimen is calculated based on the first scanning image and the second scanning image.

As may be seen from the above embodiments of the present disclosure, in this embodiment, a loading experiment is carried out on the target specimen prepared by any optional implementation manner in Embodiment 1 and Embodiment 2. By imaging the target specimen during the experiment through the target imaging equipment, a state change inside the target specimen during the experiment may be further accurately obtained through DVC, and a distribution evolution law of the displacement field and the strain field inside the target specimen may be obtained.

The specific embodiments of the present application have been described. Other embodiments are within the scope of the appended claims. In some cases, the actions recorded in the claims may be executed in a different order and can still achieve desired results. In addition, the procedures depicted in the drawings do not necessarily require a specific order or a continuous order shown to achieve the desired results. In some embodiments, multi-task processing and parallel processing may be beneficial.

The present application is described with reference to the flowcharts and/or block diagrams of the methods based on the embodiments of the present application. It should be understood that each process and/or block in the flowcharts and/or block diagrams, and the combination of processes and/or blocks in the flowcharts and/or block diagrams, may be implemented by computer program instructions. These computer program instructions may be provided to the processor of a general-purpose computer, a special-purpose computer, an embedded processor or other programmable data processing device to generate a machine, so that the instructions executed by the processor of the computer or other programmable data processing device generate a device for implementing the functions specified in one or more processes of the flowchart and/or one or more blocks of the block diagram.

It should also be noted that the terms “include”, “include” or any other variants thereof are intended to cover non-exclusive inclusion, so that a process, method, commodity or device including a series of elements not only includes those elements, but also includes other elements not explicitly listed, or also includes elements inherent to this procedure, method, commodity or device. Without more limitations, the element defined by the statement “include a . . . ” does not exclude the existence of other identical elements in the process, method, commodity or device including the element.

Those skilled in the art should understand that the present application may be implemented in the form of a completely hardware embodiment, a completely software embodiment or an embodiment combining software and hardware aspects. Moreover, the present application may be implemented in the form of a computer program product implemented on one or more computer storage media (including but not limited to a disk memory, a compact disc read-only memory (CD-ROM), an optical memory, etc.) including a computer-available program code.

Each embodiment in this specification is described in a progressive manner. The same or similar parts among the embodiments may be referred to each other, and each embodiment focuses on the differences from other embodiments.

The above are only the embodiments of the present application and are not intended to limit the present application. For those skilled in the art, the present application can have various changes and modifications. Any modifications, equivalent replacements, improvements, etc. made within the spirit and principles of the present application should be included within the scope of the claims of the present application.

1. A method for specimen preparation, comprising:
generating a plurality of first digital particle models, wherein voxel regions simulated by all the first digital particle models are of a same size, and particle radius values of speckle particles simulated by all the first digital particle models and/or volume fraction values of speckle particles simulated by all the first digital particle models are different;
generating a plurality of first volumetric images based on central point positions of the speckle particles simulated by each first digital particle model of the plurality of first digital particle models, wherein the first volumetric image is a Gaussian speckle volumetric image;
determining a simulated optimal particle radius value and a simulated optimal volume fraction value corresponding to speckle particles comprised in a target specimen based on all the first volumetric images; and
preparing the target specimen based on the simulated optimal particle radius value and the simulated optimal volume fraction value corresponding to the speckle particles comprised in the target specimen.

2. The method according to claim 1, wherein the determining a simulated optimal particle radius value and a simulated optimal volume fraction value corresponding to speckle particles comprised in a target specimen based on all the first volumetric images comprises:
performing translation transformation on all the first volumetric images separately, and calculating a root mean squared error value corresponding to each first volumetric image of the plurality of first volumetric images;
performing surface fitting on the root mean squared error values corresponding to all the first volumetric images, the particle radius values of the speckle particles and the volume fraction values of the speckle particles to obtain a first fitting surface; and
determining the simulated optimal particle radius value and the simulated optimal volume fraction value corresponding to the speckle particles comprised in the target specimen based on a minimum value of the root mean squared error values in the first fitting surface.

3. The method according to claim 2, wherein the performing translation transformation on all the first volumetric images separately, and calculating a root mean squared error value corresponding to each first volumetric image of the plurality of first volumetric images comprises:
calculating the root mean squared error value corresponding to each first volumetric image of the plurality of first volumetric images through a formula of:
$$RMSE = \sqrt{{( \frac{{\sum }_{s = 1}^{N} {d}_{s}}{N} - {d}_{0} )}^{2} + {\sum }_{s = 1}^{N} {( \frac{{\sum }_{s = 1}^{N} {d}_{s}}{N} - {d}_{s} )}^{2} / ( N - 1 )} ,$$
wherein N is a number of speckle particles in the first digital particle model, dₛ is a displacement calculation value of an s-th speckle particle calculated by Digital Volume Correlation (DVC), and d₀ is a sub-voxel displacement value of the first volumetric image.

4. The method according to claim 1, wherein the determining a simulated optimal particle radius value and a simulated optimal volume fraction value corresponding to speckle particles comprised in a target specimen based on all the first volumetric images comprises:
calculating a mean intensity gradient value corresponding to each first volumetric image of the plurality of first volumetric images;
performing surface fitting on the mean intensity gradient values corresponding to all the first volumetric images, the particle radius values of the speckle particles and the volume fraction values of the speckle particles to obtain a second fitting surface; and
determining the simulated optimal particle radius value and the simulated optimal volume fraction value corresponding to the speckle particles comprised in the target specimen based on a maximum value of the mean intensity gradient values in the second fitting surface.

5. The method according to claim 1, further comprising:
scanning, by using a target imaging equipment, a test specimen to obtain a second volumetric image, wherein a matrix of the test specimen is embedded with speckle particles with multiple particle radius values;
calculating a particle equivalent speckle size value and a noise equivalent speckle size value corresponding to the speckle particles with multiple particle radius values comprised in the test specimen based on the second volumetric image; and
obtaining a physical optimal particle radius value corresponding to the target imaging equipment based on the particle equivalent speckle size value and the noise equivalent speckle size value corresponding to the speckle particles with multiple particle radius values comprised in the test specimen,
wherein the preparing the target specimen based on the simulated optimal particle radius value and the simulated optimal volume fraction value corresponding to the speckle particles comprised in the target specimen comprises:
preparing the target specimen based on the simulated optimal particle radius value and the simulated optimal volume fraction value corresponding to the speckle particles comprised in the target specimen, and the physical optimal particle radius value corresponding to the target imaging equipment.

6. The method according to claim 5, wherein the preparing the target specimen based on the simulated optimal particle radius value and the simulated optimal volume fraction value corresponding to the speckle particles comprised in the target specimen, and the physical optimal particle radius value corresponding to the target imaging equipment comprises:
generating a second digital particle model based on a preset shape of the target specimen, and the simulated optimal particle radius value and the simulated optimal volume fraction value corresponding to the speckle particles comprised in the target specimen;
scaling the second digital particle model based on the physical optimal particle radius value corresponding to the target imaging equipment to obtain a three-dimensional (3D) digital volume model; and
preparing the target specimen with a same shape and size as the 3D digital volume model.

7. The method according to claim 6, wherein the preparing the target specimen with a same shape and size as the 3D digital volume model comprises:
generating a third volumetric image based on central point positions of all speckle particles simulated by the 3D digital volume model and the physical optimal particle radius value corresponding to the target imaging equipment, wherein in the third volumetric image, regions where the speckle particles are located and a region where the matrix is located are distinguished by binarization; and
preparing, based on the third volumetric image, the target specimen with the same shape and size as the 3D digital volume model by 3D printing.

8. The method according to claim 5, wherein the calculating a particle equivalent speckle size value and a noise equivalent speckle size value corresponding to the speckle particles with multiple particle radius values comprised in the test specimen based on the second volumetric image comprises:
performing segmentation on the second volumetric image to obtain volumetric image regions corresponding to the speckle particles with multiple particle radius values comprised in the test specimen;
performing autocorrelation analysis on the volumetric image region corresponding to the speckle particles of each particle radius value respectively after Fourier transformation, to obtain an autocorrelation curve corresponding to the speckle particles of each particle radius value; and
determining, in a case of an autocorrelation coefficient in the autocorrelation curve corresponding to the speckle particles of each particle radius value being equal to a preset coefficient value, a width of the autocorrelation curve as the particle equivalent speckle size value corresponding to the speckle particles of each particle radius value.

9. The method according to claim 5, wherein the calculating a particle equivalent speckle size value and a noise equivalent speckle size value corresponding to the speckle particles with multiple particle radius values comprised in the test specimen based on the second volumetric image comprises:
performing segmentation on the second volumetric image to obtain volumetric image regions corresponding to the speckle particles with multiple particle radius values comprised in the test specimen, wherein volumetric image regions corresponding to the speckle particles of all particle radius values are of a same size, and each volumetric image region of the volumetric image regions comprises images of speckle particles of only one particle radius value;
calculating a matrix equivalent speckle size value corresponding to a matrix in volumetric image regions respectively corresponding to the speckle particles of each particle radius value; and
obtaining the noise equivalent speckle size value corresponding to the speckle particles of each particle radius value based on the matrix equivalent speckle size value corresponding to the matrix.

10. A method for characterizing internal three-dimensional deformation of specimens, comprising:
performing scanning and imaging, by using a target imaging equipment, on the target specimen prepared by the method according to claim 1 to obtain a first scanning image;
performing a loading experiment on the target specimen to deform the target specimen;
performing scanning and imaging, by using the target imaging equipment, on the deformed target specimen to obtain a second scanning image, and
calculating a displacement field and/or a strain field inside the target specimen based on the first scanning image and the second scanning image.
